SEQUENCE LISTING (8 sequences)

SEQ ID NO: 1 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 2 - protein, 170 aa, artificial sequence
  Truncated FGF-18 (sprifermin)
MEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTK

SEQ ID NO: 3 - protein, 32 aa, artificial sequence
  calcitonin (salmon calcitonin)
CSNLSTCVLGKLSQELHKLQTYPRTNTGSGTP

SEQ ID NO: 4 - protein, 396 aa, Homo Sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 5 - protein, 431 aa, Homo Sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 6 - protein, 501 aa, Homo Sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 7 - protein, 288 aa, Homo Sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 8 - protein, 208 aa, Homo Sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

(19) United States
(12) Patent Application Publication
(10) Pub. No.: US 2022/0184180 A1
(43) Pub. Date: Jun. 16, 2022

(54) TREATMENT OF PATIENTS AT RISK OF RAPID PROGRESSION OF OSTEOARTHRITIS

(71) Applicant: MERCK PATENT GMBH, DARMSTADT (DE)

(72) Inventors: CHRISTOPH H. LADEL, DARMSTADT (DE); HANS GUEHRING, DARMSTADT (DE)

(21) Appl. No.: 17/428,995

(22) PCT Filed: Feb. 7, 2020
(86) PCT No.: PCT/EP2020/053214

§ 371 Date: Aug. 6, 2021

(30) Foreign Application Priority Data
Feb. 8, 2019 (EP) 19156281.8

(51) Int. Cl. A61K 38/18; A61P 19/02

(52) U.S. Cl. CPC A61K 38/1825; A61P 19/02

(57) ABSTRACT

The invention pertains to active compounds, in particular FGF-18 compounds, for use in the treatment of patients affected with a cartilage disorder, preferably osteoarthritis (OA), in particular for the treatment of patients who are at risk of rapid progression of the disorder.

### FIELD OF INVENTION

The invention pertains to active compounds, in particular FGF-18 compounds, for use in the treatment of patients affected with a cartilage disorder, preferably osteoarthritis (OA), in particular for the treatment of patients who are at risk of rapid progression of the disorder.

### BACKGROUND OF THE INVENTION

Cartilage disorders broadly refer to diseases characterized by degeneration of metabolic abnormalities in the connective tissues which manifest as pain, stiffness and limitation of motion of the affected body parts. These disorders can be due to pathology or can be the result of trauma or injury. Among others, cartilage disorders include osteoarthritis (OA), cartilage injury (inclusive sports injuries of cartilage and joint, and surgical injuries such as microfracture(s)). Mature cartilage has limited ability to repair itself, notably because mature chondrocytes have little potential for proliferation and due to the absence of blood vessels. In addition, cartilage is not well nitrified and has a low oxygen pressure.

OA is a progressive cartilage disorder that, at the early stage, may remain asymptomatic while the structural changes in the joint are minimal, but usually progresses towards more advanced (moderate and severe) stages. The structural changes in OA are characterized mainly by the progressive erosion and loss of articular cartilage, and the appearance or increase of symptoms of stiffness and pain. The most common way of classifying osteoarthritis is the use of the Kellgren-Lawrence (KL) grading scale, which is explained herein. Briefly the KL grading scale defines 5 stages based on radiographic analysis of the structural defects of the joint (from “0”: none, to “4”: severe).

There is not yet commercially available treatment that restores or postpones the cartilage damages (see Lotz, 2010). However, treatment options exist to manage the clinical symptoms, that will vary depending on the severity, or stage, of the disease. Treatments of the early stages involves mostly physical therapy, lifestyle modification (e.g. increasing physical activity), and supportive devices. However, as osteoarthritis progresses to minimal or moderate stages, the worsening of clinical symptoms may require the use of pain medication such as non-steroidal anti-inflammatory drugs. Those are effective in relieving osteoarthritis pain and decreasing joint swelling and inflammation, but their use may be limited by stomach irritation. In the severe or late stages, stronger pain medication may be useful, yet, in some cases surgical procedures may be necessary.

When surgical treatment is required, the standard procedure is age dependent and varies between total joint replacement, transplantation of pieces of cartilage or marrow stimulating technique (such as microfracture). Tibial or femoral osteotomies (cutting the bone to rebalance joint wear) may reduce symptoms, help to maintain an active lifestyle, and delay the need for total joint replacement. Total joint replacement can provide relief for the symptom of advanced osteoarthritis, but generally requires a change in a subject's lifestyle and/or activity level. Replacement of damaged cartilage, in particular articular cartilage, caused either by injury or disease is a major challenge for physicians, and available surgical treatment procedures are considered not completely predictable and effective for only a limited time. Microfracture is a common procedure that involves penetration of the subchondral bone to stimulate cartilage deposition by bone marrow derived stem cells. However, it has been shown that this technique does not repair sufficiently the chondral defect and the new cartilage formed is mainly fibrocartilage, resulting in inadequate or altered function and biomechanics. Indeed, fibrocartilage does not have the same durability and may not adhere correctly to the surrounding hyaline cartilage. For this reason, the newly synthesized fibrocartilage may breakdown more easily (expected time frame: 5-10 years).

Therefore, for their vast majority, younger subjects either do not seek surgical treatment or are counselled to postpone surgical treatment for as long as possible.

It is well known that disease progression is not consistent among patients suffering from knee OA and that a large number of factors are associated with a risk of rapid progression. The rate of joint space narrowing, that is to say the rate at which the thinning of the cartilage occurs, is a good indication of the progression of the disease but requires that data be collected for a certain period of time prior to making any conclusion or prognosis. Some parameters measured in clinical studies at baseline, that is to say prior to any drug administration, have been correlated with the risk of a rapid progression of the disorder. Notably, radiographic OA at baseline, defined has OA of a KL grade of 2 or more, has been associated with progression of the disorder (Guermazi et al., 2015). The joint space width (JSW) in particular in the medial compartment (mJSW), measured at baseline, is considered a strong predictive value inversely correlated with the rate of progression of knee OA (Pelletier et al., 2007). Consistently, the value of medial JSW at baseline is also a strong predictor for total knee replacement. In addition, there is evidence that knee pain not only is a consequence of structural deterioration in osteoarthritis (OA) but also contributes to structural progression. Joint pain, which may be assessed by the WOMAC Index, has further been identified as another strong predictor of structural progression of OA, and subjects having a OA of Kellgren-Lawrence grade 2 or more and experiencing persistent knee pain show an increased risk of progressive OA (Wang et al., 2018).

Those patients who are at risk of rapid progression of this cartilage disorder may not be able to avoid surgical treatment and can only find relief from pain medication for a short period of time.

There is thus a need for new therapeutic strategies, that would limit the structural progression of the disorder and ideally help with managing the increasing pain associated with OA, in particular for the treatment of patients at risk of rapid progression of cartilage disorder.

### SUMMARY OF THE INVENTION

The invention pertains to an active compound, preferably a FGF-18 compound, for use in the treatment of a subject having a cartilage disorder, wherein the subject presents with a risk of rapid progression of said cartilage disorder. As defined in more details herein, patients are considered as being at risk of a rapid progression of cartilage disorder when they present with a combination of the two following parameters: (a) significant structural defects of the joint and (b) non-acceptable joint pain.

The invention further pertains to a method for treating a subject having a cartilage disorder, comprising the steps of:
a) Determining whether said subject presents with at least a significant structural defect of at least one joint, wherein the significant structural defect is selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) Selecting the subject having:
   i. at least a significant structural defect of at least one joint, and;
   ii. a non-acceptable joint pain and;
d) Administering an active compound, preferably a FGF-18 compound, to the selected subject.

The invention further pertains to a method for selecting a subject having a cartilage disorder for inclusion in treatment, or clinical trial, with an active compound, based on the likelihood of their sensitivity to said treatment, comprising the steps of:
a) determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) selecting the sensitive subjects as being suitable for said treatment or clinical trial.

The present invention further pertains to a method of determining placebo effect in a clinical trial, preferably wherein said clinical trial is related to the treatment of a cartilage disorder in a subject with an active compound, or during a treatment of a cartilage disorder with an active compound, the method comprising the steps of:
a) determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) determining from the result of steps a) and b) the placebo effect.

### Definitions

The term “active compound” herein refers to a compound selected for instance form the group consisting of FGF-18 compound, BMP-2, BMP-7, GDF-5, FGFβ, FGF-9, SOX-9 enhancers, TGFβ, Wnt inhibitors, anti-MMP13 inhibitors, anti-ADAMTS4 or 5 inhibitors, calcitonin and any variants or fusion proteins thereof.
The term “FGF-18 compound” or “FGF-18”, as used herein, is intended to refer to a protein maintaining at least one biological activity (e.g. increase in osteoblastic activity, see WO98/1664, or in cartilage formation, see WO2008/023063) of the wildtype human FGF-18 protein. FGF-18 may be native (SEQ ID NO: 1), in its mature form (corresponding to the amino acid sequence from residue 28(Glu) to residue 207(Ala) of SEQ ID NO: 1), or a truncated form thereof such as sprifermin (as shown in SEQ ID NO:2; with amino acid residues 2 to 170 of SEQ ID NO:2 corresponding to amino acid residues 28 to 196 of SEQ ID NO:1). The term “FGF-18 compound” also includes variants or mutants of the native, mature form, or truncated forms of FGF-18, as well as fusion proteins comprising a (biologically) active FGF-18 moiety coupled to a heterologous protein or a chemical compound (such as those disclosed in EP17192467.3 patent family). In such fusion proteins, the FGF-18 moiety can be the native, mature form, or truncated forms of the FGF-18 protein or variants or mutants thereof.
The term “calcitonin” as used herein, refers to the salmon calcitonin type, a 32-amino-acid peptide (SEQ ID NO.3), which demonstrated to have protective activity on both bone and cartilage.
The term “BMP-2”, as used herein, refers to a protein inducing matrix synthesis and promoting cartilage repair as well as playing a critical role in the differentiation of osteoprogenitor cells into osteoblasts, thus promoting bone and cartilage formation (Deng et al., 2018). The full-length native form of the human BMP-2 is represented in SEQ ID NO.4. One of the recombinant forms of BMP-2 protein is known as Dibotermin alfa. This term “BMP-2” also includes variants thereof or fusion proteins comprising a BMP-2 moiety
The term “BMP-7”, as used herein, refers to a protein known for its osteogenic properties, shown to have a strong anabolic effect on cartilage by stimulating synthesis of cartilage matrix components and increasing proteoglycan and collagen synthesis (Deng et al., 2018). The full-length native form of the human BMP7 is represented in SEQ ID NO.5. One of the recombinant forms of BMP-2 protein is known as eptotermin alfa. This term also includes variants thereof or fusion proteins comprising a BMP-7 moiety
The term “GDF-5”, also known as LAP-4 or radotermin, as used herein, refers to a protein, having among others, stimulatory effects on the synthesis of matrix in human articular chondrocytes cultured in vitro, from both healthy subjects as well as OA patients (Parrish et al., 2017). The full-length native form of the human GDF-5 is represented in SEQ ID NO.6. This term also includes variants thereof or fusion proteins comprising a GDF-5 moiety.
The term “FGFβ” or “FGF-2”, as used herein, refers to a protein known in cartilage repair. It was also shown to stimulate the proliferation of chondrocytes in immature rabbits (Ameye and Young, 2006). The full-length native form of the human FGF-2 is represented in SEQ ID NO.7. One of the recombinant forms of FGFβ protein is known as trafermin. This term also includes variants thereof or fusion proteins comprising an FGFβ moiety.
The term “FGF-9”, as used herein, refers to a protein known to delay articular cartilage degradation in OA subject, while having a rather negative impact on osteophyte formation (Zhou et al., 2016). The full-length native form of the human FGF-9 is represented in SEQ ID NO.8. This term also includes variants thereof or fusion proteins comprising a FGF-9 moiety.
The term “TGF-β”, as used herein, refers to a protein TGF-beta belonging to the TGF-beta family having a crucial role in cartilage maintenance. TGF-beta has been shown as an enhancer of cartilage (Wang 2014). This term also includes variants thereof or fusion proteins comprising a TGF-β moiety.
The term “SOX-9” enhancers. as used herein, is intended to refer to a compound enhancing the production of SOX9. Indeed, SOX9 is a transcription factor shown to be essential for cartilage extracellular matrix (ECM) formation.
The term “Wnt inhibitors” as used herein, is intended to refer to a compound interfering with WNT pathway.
The term “anti-MMP13 inhibitors” as used herein is intended to refer to a compound inhibiting the activity of the matrix metalloproteinase 13 (MMP13). MMP13 is one of the key collagen type II degrading enzymes.
The term “anti-ADAMTS4 or 5 inhibitors” as used herein, is intended to refer to compounds inhibiting the enzymatic activity of a disintegrin and metalloproteinase with thrombospondin motifs 4 or 5 (ADAMTS4 or ADAMTS5).
The term “SD” means standard deviation and is linked to the usual deviations of any validation assays/systems.
The term “cartilage disorder”, as used herein, encompasses disorders resulting from damages due to injury, such as traumatic injury, chondropathy or arthritis. Examples of cartilage disorders that may be treated by the administration of the compounds described herein include but are not restricted to arthritis, such as osteoarthritis, cartilage injury, fractures affecting joint cartilage or surgical procedures with impact on joint cartilage (e.g. Microfracture). Degenerative diseases/disorders of the cartilage or of the joint, such as chondrocalcinosis, polychondritis, relapsing polychondritis, ankylosing spondylitis or costochondritis are also encompassed by this wording. The International Cartilage Repair Society has proposed an arthroscopic grading system to assess the severity of the cartilage defect: grade 0: (normal) healthy cartilage, grade 1: the cartilage has a soft spot or blisters, grade 2: minor tears visible in the cartilage, grade 3: lesions have deep crevices (more than 50% of cartilage layer) and grade 4: the cartilage tear exposes the underlying (subchondral) bone (see for instance page 13 of www.cartilage.orgLfiles/contentmanagement/ICRS_evaluation.pdf).
The term “osteoarthritis” as used herein is intended to refer to the most common forms of arthritis. The term “osteoarthritis” encompasses both primary osteoarthritis and secondary osteoarthritis (see for instance The Merck Manual, 17th edition, page 449). The most common way of classifying/grading osteoarthritis is the use of the Kellgren-Lawrence radiographic grading scale (see table below). Osteoarthritis may be caused by the breakdown of cartilage. Bits of cartilage may break off and cause pain and swelling in the joint between bones. Over time, the cartilage may wear away entirely, and the bones will rub together. Osteoarthritis can affect any joint but usually concerns hands and weight-bearing joints such as hips, knees, feet, and spine. In a preferred example, the osteoarthritis may be knee osteoarthritis or hip osteoarthritis. Osteoarthritis is one of the preferred cartilage disorders that can be treated by administering the compounds according to the present invention.

Kellgren-Lawrence Radiographic Grading Scale (KL) of Osteoarthritis is described as follow:

| Grade of | |
|---|---|
| Osteoarthritis | Description |
| 0—None | No radiographic findings of osteoarthritis |
| 1—Doubtful | Doubtful narrowing of joint space and possible |
| | osteophytic lipping |
| 2—Minimal | Definite osteophytes, definite narrowing of joint space |
| 3—Moderate | Moderate multiple osteophytes, definite narrowing of |
| | joints space, some sclerosis and possible deformity of |
| | bone contour |
| 4—Severe | Large osteophytes, marked narrowing of joint space, |
| | severe sclerosis and definite deformity of bone contour |

Grades 1 and 2 can be considered as less severe forms of the disease, whereas grades 3 and 4 can be considered as more severe forms of the disease.
The term “cartilage injury” as used herein refers to a cartilage disorder or cartilage damage resulting notably further to an accident or surgery (for instance microfracture surgery). This term “cartilage injury” also includes chondral or osteochondral fracture, damage to meniscus, and the term microfracture. Also considered within this definition is sport-related injury or sport-related wear of tissues of the joint.
The term “joint space width (JSW)” herein refers to joint space width as measured by X-ray using a standardized technique such as. fixed flexion protocol and others, as disclosed in Hunter et al., 2009. Measurement of JSW by X-ray is a recognized endpoint accepted by the European Medicines Agency and the United States Food and Drug Administration for use in efficacy studies in OA. The term “medial joint space width (mJSW)” herein refer to joint space width as measured in the medial compartment of the joint, in particular the knee, by X-ray. The term “lateral joint space width (IJSW)” herein refer to joint space width as measured in the lateral compartment of the joint, in particular the knee, by X-ray. The term “minimal joint space width (miniJSW)” herein refers to the minimal joint space width as measured in the joint in either the medial or the lateral compartment of the joint, in particular the knee, by X-ray.
The term “thin cartilage” refers to a cartilage having a JSW inferior or equal to 3.5 mm.
The term “thick cartilage” refers to a cartilage having a JSW superior to 3.5 mm.
The term “progression of cartilage disorder” as used herein refers to the increase in structural defects of the cartilage and/or joint affected by the cartilage disorder, in particular joint space narrowing (JSN), and the consequent appearance or increase in clinical symptoms such as pain, disability and joint stiffness, as a consequence of the evolution of the cartilage disorder over time. With respect to OA, the progression of the disorder may for instance be observed and assessed using the KL grading scale defined above.
The term “at risk of further structural and symptom progression of cartilage disorder” also referred to as “at risk of rapid progression of cartilage disorder”, used herein in connection with the subject to be treated, refers to a propensity of said subject to show rapid progression of cartilage disorder as a consequence of the natural evolution of the disorder over time in the absence of treatment. These terms therefore exclude structural progression of cartilage disorder that would be due to in trauma or injuries which are not consecutive to the cartilage disorder.
The term “subject” or “patient” refers to both human and non-human animals. The term non-human comprises mammals such as rodents (including mice), rabbits, cats, dogs, horses, cows, sheep, or primates.
The term “significant structural defects of the joint” herein refers to structural defects of the joint such as for instance significant minimal joint space width (miniJSW), or a a significant KL grade, and in particular a minimal joint space width (miniJSW) of less than 3.5 mm, preferably of between 1.5 mm and 3.5 mm, a KL grade of between 2 to 4, preferably a KL grade of 3. Yet preferably, the preferred significant structural defect of the joint is a minimal joint space width (miniJSW) of between 1.5 mm and 3.5 mm.
The term “non-acceptable joint pain” herein refers to a significant level of pain of the joint. Pain levels can be assessed using methods generally used in the arts and I particular in the context of clinical trials of OA patients; Such methods include but are not limited to the patient reported outcome measurement methods NRS, VAS pain, KOOS and WOMAC pain score defined hereunder.

In the context of the invention, a WOMAC pain score of 35 points or above, preferably of 40 points or above, is indicative of non-acceptable joint pain

In the context of the invention, a VAS pain score of 4 and higher (on a numeric scale) or 40 and higher (on a 100 mm scale), is indicative of non-acceptable joint pain (Williamson et al., 2005).

In the context of the invention, a NRS score of 4 and higher (on a 0-11 scale) is indicative of non-acceptable joint pain (Williamson et al., 2005).

In the context of the invention, a KOOS score of 40 and above (on a 0-100 scale), is indicative of non-acceptable joint pain (Roos et al., 2003).
The term “WOMAC Index” as used herein refers to the WOMAC® 3.1 Index (“WOMAC” for “Western Ontario and McMaster Universities Osteoarthritis Index”, 3.1 version). The Index is a self-administered questionnaire and assesses the three dimensions of pain, disability and joint stiffness in knee and hip osteoarthritis. When applied to assessing of pain and dysfunction associated with cartilage injury, it consists of a questionnaire containing 24 items (5 items for Pain, 2 items for Stiffness and 17 items for Physical Function) (see Bellamy et al., 1988; Wolfe, 1999). It is a well-known instrument, widely used notably in assessment of the OA severity. The latest version of the instrument (WOMAC® 3.1) is available in over 100 alternate language forms, and can thus easily be administered to any subject, regardless of his native language.
The term “WOMAC Total score” or “WOMAC scores” herein refers to the sum of the scores obtained by a specific patient in response to the WOMAC Index questionnaire (“WOMAC” for “Western Ontario and McMaster Universities Osteoarthritis Index”) which measures pain (WOMAC pain score) based on 5 items, function (WOMAC function score) based on 2 items and stiffness (WOMAC stiffness score) based on 17 items: Each item is rated based on the response (none=0 point, mild=1 point, moderate=2 points, severe=3 points, extreme=4 points); The total WOMAC score corresponds to the sum of the rates obtained for the 24 items; The WOMAC pain score corresponds to the sum of the rates obtained for the 5 items related to pain, optionally then normalized to a 0-100 points scale (that is to say the WOMAC pain score multiplied by 5). Preferably, in the context of the invention the WOMAC pain score indicated corresponds to the WOMAC pain score normalized to a 0-100 points scale; The WOMAC function score corresponds to the sum of the rates obtained for the 2 items related to function, optionally then normalized to a 0-100 points scale (that is to say multiplied by 100/8). Preferably, in the context of the invention the WOMAC function score indicated corresponds to the WOMAC function score normalized to a 0-100 points scale. The WOMAC stiffness score corresponds to the sum of the rates obtained for the 17 items related to function, optionally then normalized to a 0-100 points scale (that is to say multiplied by 100/68). Preferably, in the context of the invention the WOMAC stiffness score indicated corresponds to the WOMAC stiffness score normalized to a 0-100 points scale.
The term “Visual Analog Scale for Pain (VAS Pain)” herein refers to a self-administered questionnaire which is well known in the art and has been discussed in detail by Hawker et al.
The term “Numeric Rating Scale for Pain (NRS Pain)” herein refers to a self-administered questionnaire which is well known in the art and has been discussed in detail by Hawker et al.
The term “Knee Injury and Osteoarthritis Outcome Score (KOOS)” herein refers to a self-administered questionnaire which holds five separately scored subscales: pain, other symptoms, function in daily living (ADL), function in sport and recreation (Sport/Rec), and knee-related quality of life (QOL). Preferably, the terms refer to the use of a questionnaire available in different languages as described in Roos et al. 1998, Roos et al. 2003 Collins et al. 2016
The term “cartilage thinning” refers to the decrease in cartilage volume and/or thickness over time as a consequence of the evolution of the cartilage disorder in the absence of treatment. In the context of the invention, cartilage thinning may be assessed by measuring cartilage thickness using magnetic resonance imaging (MRI) measurements, including Lateral volume of cartilage (also referred as LFTC), Medial volume of cartilage (also referred as MFTC), Total volume of cartilage (also referred as LFTC+MFTC) and new total average cartilage thickness, at different time points.
The term “limit cartilage thinning associated with a cartilage disorder”, with regards to the therapeutic effect of FGF-18 compound, refer to the diminution of cartilage thinning over time in a subject treated with said compound, compared to cartilage thinning occurring or likely to have occurred over time in the absence of treatment. The cartilage thinning occurring or likely to have occurred over time in the absence of treatment can be estimated for instance based on results of clinical trials.
The term “prevent cartilage thinning associated with a cartilage disorder”, when describing the therapeutic effect of FGF-18 compound, refers to the inhibition of cartilage thinning over time in a subject treated with said compound, compared to the cartilage thickness of the subject prior to said treatment.
The term “clinical symptoms associated with a cartilage disorder” herein refers to clinical symptoms such as pain, disability and joint stiffness, resulting from the cartilage disorder. Clinical symptoms associated with a cartilage disorder, and those associated with the evolution of the cartilage disorder, may be assessed using the WOMAC Index as defined herein. Pain can be assessed by the WOMAC pain score, and a WOMAC pain score of 20 or above is indicative of moderate to severe pain, while a WOMAC pain score of 35 or above is indicative of non-acceptable pain (Goggins et al. 2005). Similarly, disability and joint stiffness can be assessed by the WOMAC function and WOMAC stiffness score respectively.
The term “clinical symptoms associated with the evolution of a cartilage disorder over time” herein refers to the symptoms arising over time as a result of the natural evolution of the cartilage disorder in the absence of treatment, and include increased pain, increased disability and increased joint stiffness. An increase of the WOMAC index overtime is indicative that the clinical symptoms are increasing. In particular, an increase of the WOMAC pain score of a subject over time is indicative that pain is increasing. Similarly, an increase of the WOMAC function and WOMAC stiffness score over time is an indication that disability and joint stiffness are increasing respectively.
The term “limit the clinical symptoms associated with the cartilage disorder” and “limit the clinical symptoms associated with the evolution of a cartilage disorder over time”, with regards to the therapeutic effect of FGF-18 compound, refers to the diminution of the clinical symptoms as defined above over time in a subject treated with said compound, compared to clinical symptoms in the absence of treatment.
The term “SD” means standard deviation and is linked to the usual deviations of any validation assays/systems.
The term “placebo” herein refers to a compound or composition devoid of any therapeutic activity.
The term “placebo effect” as used herein is to be understood as changes in structural defects or clinical symptoms, compared to baseline, that is to say compared to the structural defects or clinical symptoms in the absence of any administration, due to the administration of a placebo. The term “low placebo effect” refers to a response magnitude comparable or only minimal change (below 20%) to the one at baseline, within the standard deviation of the assessment method. The term “strong placebo effect” as used herein is to be understood as a change by more than 20% from baseline:

### DETAILED DESCRIPTION OF THE INVENTION

The surprising finding of the present invention is based on different studies aimed at identifying potential subgroups associated with a different response to therapy. The parameters used in these studies were composed of imaging techniques and patient reported outcome measures such as the WOMAC scores. JSW measurement was used as an imaging marker of the structural defects of the joint. The association between the patient reported outcome measures and/or an imaging marker like JSW and variation in the clinical symptoms was assessed. The rationale behind this type of analysis was to identify combination of markers that could be predictive of 1) placebo response and/or 2) the clinical outcome (notably with regard to cartilage repair and symptom improvement), for a subject to be treated with an active compound such as an FGF-18 compound, BMP-2, BMP-7, GDF-5, FGFβ, FGF-9, SOX-9 enhancers, TGFβ, Wnt inhibitors, anti-MMP13 inhibitors, anti-ADAMTS4 or 5 inhibitors, calcitonin and any variants or fusion proteins thereof. In particular, it was surprisingly found that the combination of structural defects and level of pain could be used to predict placebo effect (see experimental part and FIGS. 2 and 3).

The invention is based on findings that, among the variety of subjects affected with OA, and in particular knee OA, those who are at risk of further structural and symptom progression of cartilage disorder, that is to say at risk of a rapid progression of cartilage disorder, show a particularly good response to treatment with an active compound in particular a FGF-18 compound.

As defined in more details herein, patients are considered as being at risk of a rapid progression of cartilage disorder when they present with a combination of the two following parameters: (a) significant structural defects of the joint and (b) non-acceptable joint pain.

Fibroblast Growth factor 18 (FGF-18) is a member of the FGF family of proteins, closely related to FGF-8 and FGF-17. It has been shown that FGF-18 is a proliferative agent for chondrocytes and osteoblasts (Ellsworth et al., 2002; Shimoaka et al., 2002; Gigout et al., 2017). FGF-18 has been proposed for the treatment of cartilage disorder such as osteoarthritis and cartilage injury either alone (WO2008/023063) or in combination with hyaluronic acid (WO2004/032849).

Sprifermin, a truncated form of human FGF-18, is being investigated in clinical trials for treatment of both osteoarthritis and cartilage injury (see for instance NCT01033994, NCT00911469 and NCT01066871). The current dosing regimen for sprifermin is once weekly for 3 weeks (one treatment cycle), the drug being administered via intraarticular injections. This treatment cycle can be repeated. This dosing regimen has been described in WO2008/023063. Quite interestingly, in the subgroup of subjects at risk of a rapid progression of the cartilage disorder, herein also referred to as subgroup at risk, subjects at risk or patients at risk, treatment with a FGF-18 compound has been shown to limit, or even inhibit, the progression of cartilage thinning, as well as to limit the clinical symptoms associated with said cartilage disorder, in particular pain.

Interestingly, even 18 months after the last administration of treatment, patients from the subgroup at risk treated with FGF-18 show an improvement of their clinical symptoms, in particular pain, compared to the last injection time point. In other terms, even after cessation of treatment, the clinical outcomes of subjects the subgroup at risk treated with FGF-18 compound keep improving. In contrast, during the same period of time, subjects from the subgroup at risk treated with placebo show a worsening, or increase, of their clinical symptoms, in particular pain, which suggests that the FGF-18 compound improves significantly the clinical outcome in the subjects at risk. Overall, the therapeutic effects obtained with FGF-18 compound in the subgroup of subjects at risk as defined herein, seem to define a specific clinical situation that had not been investigated before.

The invention pertains to an active compound for use in the treatment of a subject having a cartilage disorder, wherein the subject presents with a risk of rapid progression of said cartilage disorder.

In the context of the invention, the active compound is selected from the group consisting of an FGF-18 compound, BMP-2, BMP-7, GDF-5, FGFβ, FGF-9, SOX-9 enhancers, TGFβ, Wnt inhibitors, anti-MMP13 inhibitors, anti-ADAMTS4 or 5 inhibitors, calcitonin and any variants or fusion proteins thereof;

Preferably, the active compound is an FGF-18 compound as defined herein.

In the context of the invention, the subject is considered as presenting with a risk of rapid progression of said cartilage disorder when said subject presents with a combination of the two following parameters:

(a) significant structural defects of the joint and;

(b) non-acceptable joint pain.

In the context of the present invention, the preferred significant structural defect of the joint is selected from the group consisting of a minimal joint space width (miniJSW) of less than 3.5 mm, preferably of between 1.5 mm and 3.5 mm, and a KL grade of between 2 to 4, preferably a KL grade of 3. Yet preferably, the preferred significant structural defect of the joint is a minimal joint space width (miniJSW) of between 1.5 mm and 3.5 mm.

In the context of the invention, the preferred non-acceptable joint pain is selected from the group consisting of a joint pain corresponding to a WOMAC pain score of at least 35 points, preferably of at least 40 points, a joint pain corresponding to a VAS pain score of 4 and higher (on a numeric scale) or 40 and higher (on a 100 mm scale), a joint pain corresponding to a NRS score of 4 and higher (on a 0-11 scale) and a joint pain corresponding to a KOOS score of 40 and above (on a 0-100 scale)

Preferably, the subject is considered as presenting with a risk of rapid progression of said cartilage disorder when said subject presents with:

(a) significant structural defects of the joint selected from the group consisting of a minimal joint space width (miniJSW) of less than 3.5 mm, preferably of between 1.5 mm and 3.5 mm, a KL grade of between 2 to 4, preferably a KL grade of 3, and;

(b) a joint pain corresponding to a WOMAC pain score of at least 35 points, preferably at least 40 points.

More preferably, the subject is considered as presenting with a risk of rapid progression of said cartilage disorder when said subject presents with:

(a) a minimal joint space width (miniJSW) of less than 3.5 mm, preferably of between 1.5 mm and 3.5 mm,

(b) a joint pain corresponding to a WOMAC pain score of at least 35 points, preferably at least 40 points.

Yet more preferably, the subject is considered as presenting with a risk of rapid progression of said cartilage disorder when said subject presents with:

(a) a minimal joint space width (miniJSW) of between 1.5 mm and 3.5 mm,

(b) a joint pain corresponding to a WOMAC pain score of at least 40 points.

In a preferred embodiment, the invention pertains to a FGF-18 compound for use in the treatment of a subject having a cartilage disorder, wherein the subject presents with

(a) a minimal joint space width (miniJSW) of between 1.5 mm and 3.5 mm,

(b) a joint pain corresponding to a WOMAC pain score of at least 40 points.

The invention further pertains to a method for treating a subject having a cartilage disorder, comprising the steps of:
a) Determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) Selecting the subject having:
   i. at least a significant structural defect of at least one joint, and
   ii. a non-acceptable joint pain and;
d) Administering an active compound, preferably a FGF-18 compound, to the selected subject.

In the context of the invention, a WOMAC pain score of 35 points or above, preferably of 40 points or above, is indicative of non-acceptable joint pain

In the context of the invention, a VAS pain score of 4 and higher (on a numeric scale) or 40 and higher (on a 100 mm scale), is indicative of non-acceptable joint pain.

In the context of the invention, a NRS score of 4 and higher (on a 0-11 scale) is indicative of non-acceptable joint pain.

In the context of the invention, a KOOS score of 40 and above (on a 0-100 scale), is indicative of non-acceptable joint pain.

Preferably, the invention pertains to a method for treating a subject having a cartilage disorder, comprising the steps of:
a) Determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and;
b) obtaining an assessment of the level of joint pain of the subject, wherein the level of joint pain is preferably assessed based on the WOMAC pain score;
c) Selecting the subject having:
   i. at least a significant structural defect of at least one joint, and
   ii. a non-acceptable joint pain and;
d) Administering a FGF-18 compound to the selected subject.

Preferably, in the context of the invention, the active compound, preferably an FGF-18 compound for use or in the method of treatment as defined above limits or prevents the progression of cartilage thinning associated with said cartilage disorder. Preferably, in the context of the invention, the active compound, preferably an FGF-18 compound for use or in the method of treatment as defined above limits or prevents the clinical symptoms associated with said cartilage disorder. Preferably the clinical symptoms are selected from the list consisting of pain, disability and joint stiffness associated with said cartilage disorder. Yet preferably, the clinical symptom is pain associated with said cartilage disorder. In a preferred embodiment, the clinical symptoms are selected from the list consisting of increasing pain, disability and joint stiffness associated with the evolution of said cartilage disorder. Yet preferably, the clinical symptom is increasing pain associated with the evolution of said cartilage disorder. Preferably, in the context of the invention, the active compound, preferably an FGF-18 compound, for use or in the method of treatment as defined above limits or prevents the progression of cartilage thinning of the subject and the clinical symptoms associated with said cartilage disorder.

In another aspect, the invention pertains to an active compound, preferably an FGF-18 compound for use in the prevention or treatment of clinical symptoms associated with a cartilage disorder in a subject having said cartilage disorder, wherein the subject presents with a risk of rapid progression of said cartilage disorder. In a preferred embodiment, the clinical symptoms are selected from the list consisting of pain, disability and joint stiffness associated with said cartilage disorder. Yet preferably, the clinical symptom is pain associated with said cartilage disorder. In a preferred embodiment, the clinical symptoms are selected from the list consisting of increasing pain, disability and joint stiffness associated with the evolution of said cartilage disorder. Yet preferably, the clinical symptom is increasing pain associated with the evolution of said cartilage disorder.

In the context of the present invention, the preferred cartilage disorder is selected from the group consisting of osteoarthritis, cartilage injury, fractures affecting joint cartilage or surgical procedures with impact on joint cartilage, such as microfracture. Advantageously, the cartilage disorder is osteoarthritis, preferably knee or hip osteoarthritis.

Preferably, the FGF-18 compound selected from the group consisting of the native FGF-18 form (SEQ ID NO: 1), native FGF-18 in its mature form (corresponding to the amino acid sequence from residue 28(Glu) to residue 207(Ala) of SEQ ID NO: 1), a truncated form of FGF-18 such as sprifermin, also designated herein as FGF-18(170AA), (as shown in SEQ ID NO:2; with amino acid residues 2 to 170 of SEQ ID NO:2 corresponding to amino acid residues 28 to 196 of SEQ ID NO:1) More preferably, the FGF-18 compound of the invention is selected from the group consisting of a) a polypeptide comprising or consisting of the human FGF-18 mature form comprising residues 28-207 25 of SEQ ID NO:1, or b) a polypeptide comprising or consisting of FGF-18(170AA)(SEQ ID NO:2).

Preferably, the FGF-18 compound is administered intraarticularly.

The FGF-18 compound should be administered at an effective dose, and according to the appropriate dosing regimen, which may be adapted by the physician according to the subject, taking for instance into consideration the gender, age, KL grade, or other parameters specific of the subject.

In a preferred embodiment the FGF-18 compound is administered at a dose of 1-100 μg, or preferably 1-60 microgram (μg), or preferably 3-50 μg, or preferably 5-40 μg, or preferably 10-30 μg per single intra-articular administration of the FGF-18 compound. In a preferred embodiment the treatment comprises administration at a dose of about 3, 5, 10, 15, 20, 25, 30, 35, 40, 45, 50, 55, 60 μg per single intra-articular administration of the FGF-18 compound. Preferred doses include 5, 10, 15, 20, 25 and 30 μg per single intra-articular administration of the FGF-18 compound.

In a further preferred embodiment, the FGF-18 compound is administered at a dose of 50-200 mcg/kg, preferably 80-120 mcg/kg per single intravenous administration of the FGF-18 compound. In a preferred embodiment the treatment comprises administration at a dose of 80, 90, 100, 110 or 120 mcg/kg per single intravenous administration of the FGF-18 compound. Preferably the FGF-18 compound is administered according to a dosing regimen comprising at least a treatment cycle of at least 2 administrations, said 2 administrations being separated by about 4, 5, 6, 7, 8, 9 or 10 days, preferably 7 days. Preferably, the dosing regimen comprises at least two treatment cycles of at least 2 administrations, said treatment cycles being separated by about 4, 5, 6, 7, 8, 9, 10, 11 or 12 months, preferably 6 months.

In a preferred embodiment, the FGF-18 compound is administered intraarticularly, at a dose of 100 μg per injection, once weekly for 3 weeks per treatment cycle, in a dosing regimen comprising at least two treatment cycles, said treatment cycles being separated by about 10 to 14 months, preferably 12 months. In a yet preferred embodiment, the FGF-18 compound is administered intraarticularly, at a dose of 100 μg per injection, once weekly for 3 weeks per treatment cycle, in a dosing regimen comprising at least four treatment cycles, said treatment cycles being separated by about 4 to 8 months, preferably 6 months.

FGF-18 compounds may be formulated as a pharmaceutical composition, i.e. together with a 20 pharmaceutically acceptable carrier, excipients or the like. The definition of “pharmaceutically acceptable” is meant to encompass any carrier, excipients or the like, which does not interfere with effectiveness of the biological activity of the active ingredient and that is not toxic to the patient to which it is administered. For example, for parenteral administration, the active protein(s) may be formulated in a unit dosage form for injection in vehicles such as saline, dextrose solution, serum 25 albumin and Ringer's solution. Formulations for intraarticular application will comply with most of the requirements that also apply to other injection formulations, i.e., they need to be sterile and compatible with the physiological conditions at the application site (e.g., knee joint, synovial fluid). The excipients used for intraarticular injection may also be present in other injection formulations, e.g., for intramuscular or subcutaneous application. Such formulations of FGF-18 compounds, including at 30 least one further pharmaceutically acceptable carrier, excipients or the like, are herein also referred to as “FGF-18 compositions” or “FGF-18 formulations”. Said “FGF-18 compositions” or “FGF-18 formulations” are also useful in the context of the present invention.

The invention further pertains to a method for selecting a subject having a cartilage disorder for inclusion in treatment, or clinical trial, with an active compound, based on the likelihood of their sensitivity to said treatment, comprising the steps of:
a) determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) selecting the sensitive subjects as being suitable for said treatment or clinical trial.

Preferably, according to said method, the presence of:
a) a significant structural defect selected from the group consisting of a minimal joint space width (miniJSW) of less than 3.5 mm, preferably of between 1.5 mm and 3.5 mm, and a KL grade of 2 to 4, preferably a KL grade of 3, and
b) non-acceptable joint pain selected from the group consisting of a joint pain corresponding to a WOMAC pain score of at least 35 points, preferably of at least 40 points, a joint pain corresponding to a VAS pain score of 4 and higher (on a numeric scale) or 40 and higher (on a 100 mm scale), a joint pain corresponding to a NRS score of 4 and higher (on a 0-11 scale) and a joint pain corresponding to a KOOS score of 40 and above (on a 0-100 scale),

is indicative that the subject is sensitive to said treatment.

The present invention further pertains to a method of determining placebo effect in a clinical trial, preferably wherein said clinical trial is related to the treatment of a cartilage disorder in a subject with an active compound, or during a treatment of a cartilage disorder with an active compound, the method comprising the steps of:
a) determining whether said subject presents with at least a significant structural defect of at least one joint, wherein the significant structural defect is preferably selected from the group consisting of a minimal joint space width (miniJSW) of less than 3.5 mm, preferably of between 1.5 mm and 3.5 mm, and a KL grade of 2 to 4, preferably a KL grade of 3, and;
b) obtaining an assessment of the level of joint pain of the subject, wherein the level of joint pain is preferably assessed based on the WOMAC pain score, the VAS pain score, the NRS score or the KOOS score;
c) determining from the result of steps a) and b) the placebo effect.

Preferably, according to said method, the presence of:
c) a significant structural defect selected from the group consisting of a minimal joint space width (miniJSW) of less than 3.5 mm, preferably of between 1.5 mm and 3.5 mm, and a KL grade of 2 to 4, preferably a KL grade of 3, and
d) non-acceptable joint pain selected from the group consisting of a joint pain corresponding to a WOMAC pain score of at least 35 points, preferably of at least 40 points, a joint pain corresponding to a VAS pain score of 4 and higher (on a numeric scale) or 40 and higher (on a 100 mm scale), a joint pain corresponding to a NRS score of 4 and higher (on a 0-11 scale) and a joint pain corresponding to a KOOS score of 40 and above (on a 0-100 scale),

is predictive of low placebo effect.

Yet preferably, according to said method, the presence of a minimal JSW superior to 3.5 mm and WOMAC pain score inferior to 35 points, preferably inferior to 40 points, is predictive of strong placebo effect. On the contrary, the presence of a minimal JSW inferior or equal to 3.5 mm and WOMAC pain score inferior to 35 points, preferably inferior to 40 points, is predictive of no or low placebo effect.

### DESCRIPTION OF THE FIGURES

FIG. 1: Scheme of the dosing regimens used for FGF-18 compound in the FORWARD study.

FIG. 2: Observed mean difference in WOMAC pain scores between patients treated with sprifermin and placebo among different subgroups, at year 2 and at year 3 of the FORWARD study.

FIG. 3: Change in total WOMAC scores between patients treated with sprifermin and placebo among different subgroups, at year 3 of the FORWARD study.

FIG. 4: Evolution of Total MRI Cartilage Thickness (tCT) in subjects treated with different dose regimen of FGF-18 compound versus placebo, during (weeks 26, 52, 78, 104) and after (weeks 156) treatment, in the overall FORWARD study (A), and in patients at risk of developing rapid OA (B). A: Evolution of Total MRI Cartilage Thickness in the overall FORWARD study. B: Evolution of Total MRI Cartilage Thickness in the subjects presenting with a minimal joint space width in the whole knee (indicated in the figures as mJSW) of between 1.5 and 3.5 mm and a WOMAC Pain score of 40-90 points (N=171).

FIG. 5: Evolution of assessment of pain and function using WOMAC Total score in subjects treated with different dose regimen of FGF-18 compound versus placebo, during (weeks 26, 52, 78, 104) and after (weeks 156) treatment, in the overall FORWARD study (A), and in specific patients subgroups (B). A: WOMAC Total score in the overall FORWARD study B: WOMAC Total score in the subjects presenting with a minimal joint space width in the whole knee (indicated in the figures as mJSW) of between 1.5 and 3.5 mm and a WOMAC Pain score of 40-90 points.

FIG. 6: Treatment with FGF-18 compound has a marked and increased effect on pain and function in subjects at risk during treatment. Observed mean difference in WOMAC Total score in subjects after treatment with FGF-18 compound (with a regimen of FGF-18 compound:100 μg×4) versus placebo in the overall FORWARD study (ITT, for Intention To Treat), in the subgroup of subjects presenting with a WOMAC Pain score of 40 or above (independent of other criteria), in the subgroup of subjects presenting with a minimal superior to 3.5 mm (independent of other clinical criteria), and in the subgroup of subjects presenting with a minimal joint space width in the whole knee (indicated in the figures as mJSW) of between 1.5 and 3.5 mm and a WOMAC Pain score of 40-90 points.

FIG. 7: The effect on pain and function of the treatment with FGF-18 compound in subjects at risk is retained at least one year after cessation of treatment (one year after the last cycle of injections). Observed mean difference in WOMAC Total score in subjects one year after cessation of treatment (one year after the last administration of the FGF-18 compound) with FGF-18 compound (with a regimen of FGF-18 compound:100 μg×4) versus placebo in the overall FORWARD study (ITT, for Intention To Treat), in the subgroup of subjects presenting with a WOMAC Pain score of 40 or above (independent of other clinical criteria), in the subgroup of subjects presenting with a minimal joint space width in the whole knee (indicated in the figures as mJSW) superior to 3.5 mm (independent of other clinical criteria), and in the subgroup of subjects presenting with a minimal joint space width in the whole knee (indicated in the figures as mJSW) of between 1.5 and 3.5 mm and a WOMAC Pain score of 40-90 points.

### DESCRIPTION OF THE SEQUENCES

SEQ ID NO.1: Amino acid sequence of the native human FGF-18.

SEQ ID NO.2: Amino acid sequence of the recombinant truncated FGF-18 (trFGF-18).

SEQ ID NO.3: Amino acid sequence of the salmon calcitonin.

SEQ ID NO.4: Amino acid sequence of the human BMP-2

SEQ ID NO.5: Amino acid sequence of the human BMP-7

SEQ ID NO.6: Amino acid sequence of the human GDF-5.

SEQ ID NO.7: Amino acid sequence of the human FGFβ.

SEQ ID NO.8: Amino acid sequence of the human FGF-9.

### EXAMPLES

Statistical Methods

The treatment effect on the primary endpoint was assessed through dose-ranging using a repeated measurement analysis of variance (ANOVA, using PROC MIXED in SAS) on absolute change from Baseline, including the baseline value, the treatment group, the time, and the country as factors and treatment-by-time point as interaction. The primary efficacy analysis consisted of testing the linear dose relationship and the overall treatment effect at 2 years. The significance level was set at 5% 2-sided for both tests. Pairwise comparisons (sprifermin versus placebo, and between sprifermin dose and regimen groups) were performed within the context of this modelling framework. For each pairwise comparison, the difference between treatments and the corresponding 95% confidence interval (CI) and p-value are presented. The same ANOVA model used for the primary endpoint was used to assess the treatment effect on continuous secondary endpoints such as MRI endpoints, WOMAC endpoints (total, pain, function, and stiffness scores), and X-ray endpoints at each time point and over time. Logistic regression was used to assess the treatment effect on the binary efficacy endpoints such as the OMERACT-OARSI responder rate. Point estimates for each pairwise comparison and corresponding 95% CIs and p-values are provided.

Pain and Function Assessments

The WOMAC is a validated instrument used to assess symptom modification in clinical OA studies. This clinical score was developed in 1981 and is regarded as a valid instrument by both clinical researchers and regulatory authorities. The WOMAC is widely used in clinical studies in hip and knee OA and has been extensively validated.

Subjects had to answer all of the 24 questions themselves (i.e. 5 for pain, 2 for stiffness and 17 for physical function assessment), using either the 11-box NRS assessment (with categories of 0 to 10) with reference to the past 48 hours for example 1 or 100 mm VAS (visual analogue scales; giving each question a score from 0 to 100) with reference to the past 24 hours for example 2. Different forms of the questionnaire exist for the right and the left knees: in order to reduce confounding of WOMAC responses by symptoms in the contralateral knee, subjects used the WOMAC questionnaire specific to the target knee.

For administration of the questionnaire, instructions for the WOMAC 3.1 Index were followed for both examples 1 and 2.

Other instruments for assessment of pain and function are the KOOS (Knee injury and Osteoarthritis Outcome Score, Collins et al. 2016).

X-Ray Assessment of JSW

Change in JSW as measured by X-ray is a recognized endpoint accepted by the European Medicines Agency and the United States Food and Drug Administration for use in efficacy studies in OA. The JSW was measured using standardized technique. X-ray was also used to assess KL grade.

qMRI Assessment

The primary endpoint for the DBPC treatment phase was the change from Baseline in cartilage thickness in the total femorotibial joint as evaluated by qMRI at 2 years in the mITT. Cartilage thickness of the total femorotibial joint were calculated in 2 ways:
1. Average Cartilage Thickness (Total Volume divided by Total Surface Area),
2. Total Cartilage Thickness (sum of cartilage thickness in medial and lateral compartment).

The treatment effect on the primary endpoint was assessed through dose-ranging using a repeated measurement analysis of variance (ANOVA) on absolute change from Baseline, including the treatment group, the time point, and the (pooled) country as fixed factors and the baseline value as covariate and treatment by time point as interaction. Repeated measures over time were accounted for using an “unstructured” covariance pattern.

Pairwise comparisons of absolute change from Baseline in cartilage thickness (treatment with compound groups versus placebo) were performed within the context of the modelling framework described above. For each pairwise comparison, the difference between treatments and the corresponding 95% confidence interval (CI) and p-value are presented. P-values (corresponding to Type 3 tests of fixed effects) are reported for all covariates in the original “Overall” model for all time points combined (i.e., baseline value, treatment, time point, treatment-by-time point interaction, country) and for all time points. Estimated coefficients, p-values, and 95% CIs are presented overall and at each time point for (i) the dose relationship (linear trend) and (ii) each pairwise comparison between dose level and placebo. In order to assess the robustness of the primary results, the tests for linear dose-relationship and for the overall treatment effect were repeated using the PP Analysis Set. For the mITT Analysis Set, a non-parametric analysis was conducted for the ordered data of cartilage thickness in the total femorotibial joint as an alternative method for the primary analysis. Data were ordered by the magnitude of absolute change-from-Baseline over 2 years during DBPC treatment phase using rank transformation.

### Example 1. Clinical Efficacy in Subjects Treated with an FGF-18 Compound on Total Cartilage Thickness and Pain and Function as Measured by MRI and WOMAC Total Scores

The FGF-18 compound used as a treatment in the present examples is sprifermin (as defined in the section “definitions”). Two strengths of sprifermin were supplied for the study: 30 μg and 100 μg. Sprifermin was supplied as a white, sterile, freeze-dried powder in 3-mL glass vials. Each vial contained either 31.5 μg or 105 μg of sprifermin active substance; these quantities included a 5% overage, permitting extraction of respectively 30 μg or 100 μg of sprifermin active substance following reconstitution with 0.9% w/v Sodium Chloride Injection (referred to herein as “saline solution”). Excipients of the formulation were sodium phosphate buffer (pH 7.2), sodium hydroxide, O-phosphoric acid, sucrose, and poloxamer 188. For all treatment groups, the volume administered was 2 mL.

The present study was based on the FORWARD study (see study EMR700692-006).

The study enrolled adult subjects of either sex with primary femorotibial OA according to American College of Rheumatology (ACR) clinical and radiographic criteria who had Kellgren-Lawrence grades (KLG) of 2 or 3 and a minimum joint space width (JSW) of 2.5 mm in the whole knee. Subjects must have had pain in the target knee on most days and/or require symptomatic treatment of knee pain with paracetamol (acetaminophen), systemic non-steroidal anti-inflammatory drugs (NSAIDs) including COX inhibitors (COXibs), or tramadol on most days of the previous month, and must have had both: 1) A history of pain due to OA in the target knee for at least 6 months, and 2) Pain score for the target knee of 4 to 9 points in response to Question 1 of the Western Ontario and McMaster Universities Osteoarthritis Index (WOMAC) pain index (“how much pain have you had [in the target knee, over the past 48 hours] when walking on a flat surface?”) at screening and Baseline, after washout of at least 5 half-lives of analgesic medication(s): acetaminophen, topical or oral systemic NSAIDS, COXibs, opioids, and/or tramadol. Women of childbearing potential must have used a form of contraception with a failure rate of less than 1% per year throughout the study.

Main exclusion criteria included malalignment of >5 degrees in the femorotibial axis of the target knee, clinical signs of inflammation (i.e. redness) in the target knee, intraarticular. administration of corticosteroids or hyaluronic acid into either knee within 6 months before screening, any plan for knee surgery (affecting either the target or the contralateral knee) within the next 2 years, concomitant conditions or treatments deemed to be incompatible with study participation, contraindications to MRI scanning (including inability to fit in the scanner or knee coil), pregnancy or breastfeeding, participation in another clinical study within the past 30 days, and legal incapacity or limited legal capacity.

Written informed consent must have been obtained prior to any study-related activity.

Where five groups of patients were studied:
Group 1 (4 cycles placebo; hereafter referred to as placebo or PBO): 108 subjects.
Group 2 (2 cycles sprifermin 30 μg/injection alternating with 2 cycles placebo; hereafter referred to as sprifermin/placebo 30 μg×2): 110 subjects.
Group 3 (4 cycles sprifermin 30 μg/injection; hereafter referred to as sprifermin 30 μg×4): 111 subjects.
Group 4 (2 cycles sprifermin 100 μg/injection alternating with 2 cycles of placebo; hereafter referred to as sprifermin/placebo 100 μg×2): 110 subjects.
Group 5 (4 cycles sprifermin 100 μg/injection; hereafter referred to as sprifermin 100 μg×4): 110 subjects.

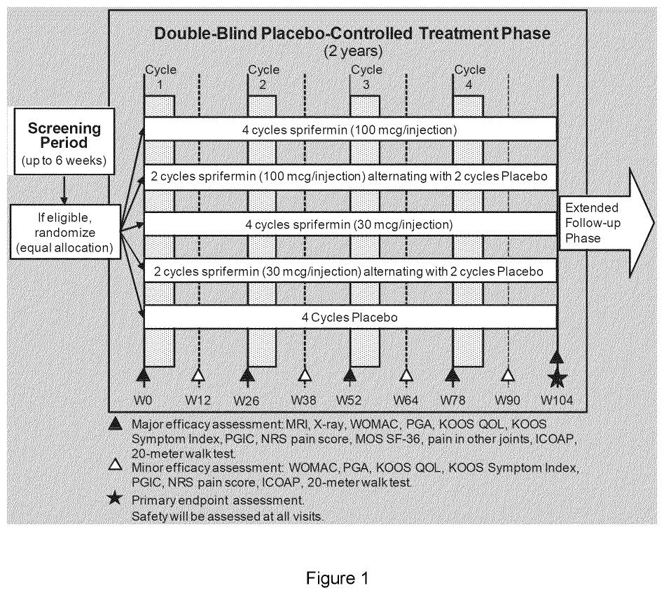

According to the FORWARD study, the patients received 4 cycles of treatment (each consisting of 3 once-weekly intra articular injections over 3 consecutive weeks) at intervals of 6 months (see FIG. 1). All injections were intraarticular (done intraarticularly).

The primary efficacy endpoint was the change from Baseline in cartilage thickness in the total femorotibial joint as evaluated by MRI at week 104 (2 years).

Exploratory endpoints included response to treatment or disease progression (response assessed by MRI and/or WOMAC index questionnaire).

Sprifermin Effect on WOMAC Pain in Different Subpopulations of Patients Based on Different Parameters at Baseline Included in the Study:

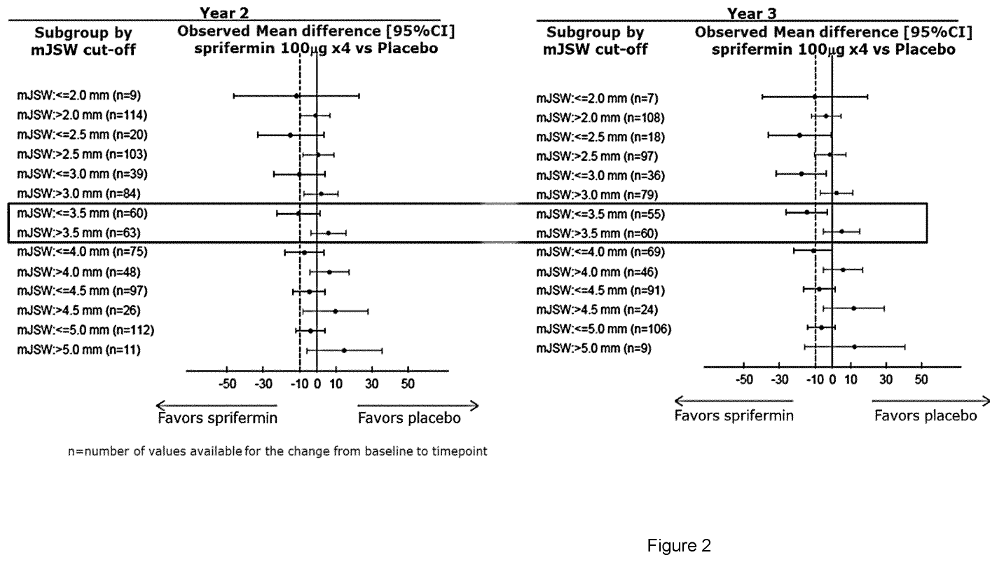

As is apparent in FIG. 2 subjects treated with sprifermin and subgrouped based on different measures at baseline experienced a different response on symptoms as determined by WOMAC pain measure. In all the figures, the term mJSW refers to the minimal joint space width in the whole knee.

Sprifermin Effect on WOMAC Pain in Different Subpopulations of Patients Based on JSW Included in the Study:

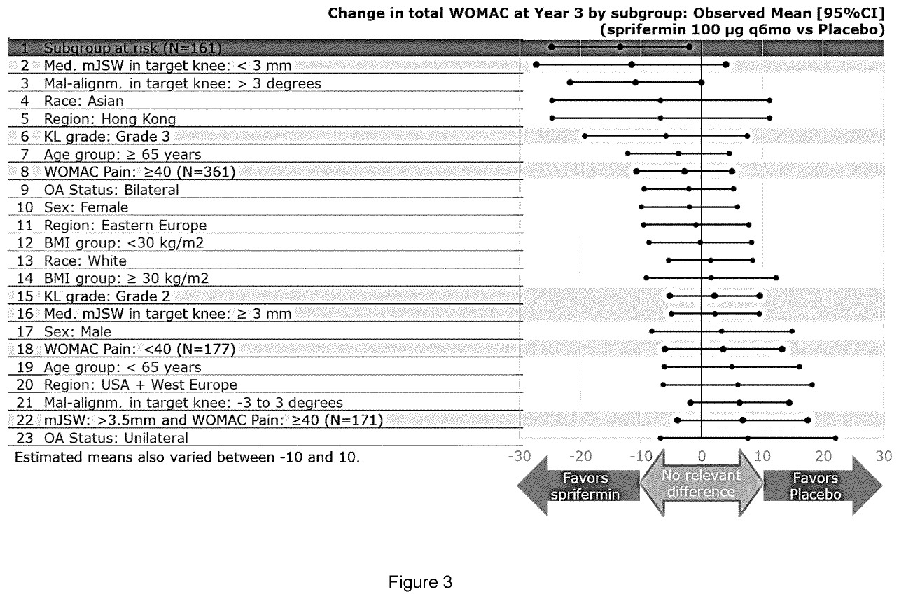

As is apparent in FIG. 3 subjects treated with sprifermin and subgrouped based on different measures experienced a different response on symptoms as determined by WOMAC pain measure. Patients with higher minimal JSW have responses in favour of placebo. In contrast patients with a minimal JSW of between 1.5±2SD mm and 3.5±2SD mm experienced a positive pain relief as indicated by a decreased WOMAC pain score. The subgroup at risk (line 1) is given a mean effect that is the most in favour of sprifermin. In all the figures, the term mJSW refers to the minimal joint space width in the whole knee.

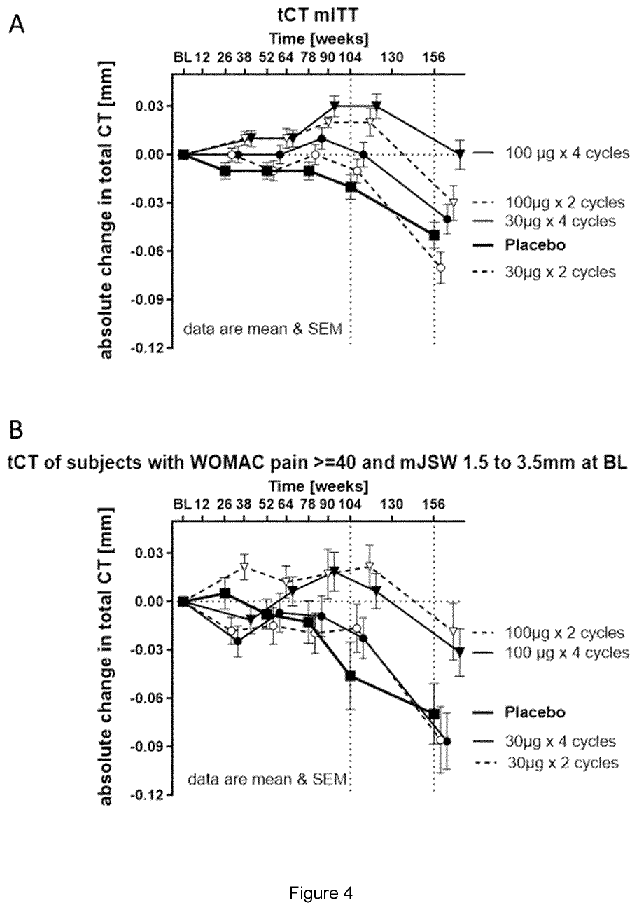

Placebo and sprifermin effect on cartilage thickness on the overall population of patients included in the study: As is apparent in FIG. 4A subjects treated with placebo experienced loss of cartilage thickness over the course of the study during the first 18 months when injections of placebo were made, and 18 months after the last injection (In contrast, subjects treated with sprifermin injections (sprifermin 100 μg×4) experienced an increase in cartilage thickness during the period of treatment Although cartilage thickness decreases after the last injection of sprifermin compound in these subjects the loss of cartilage remains significantly lower in the subjects treated with the FGF-18 compound as compared to the placebo-treated subjects over the entire length of the study (0.05 mm, p value 0.025), thus showing a limitation of cartilage thinning in all subjects treated with FGF-18.

Placebo and sprifermin effect on cartilage thickness on subjects at risk (minimal JSW of between 1.5 and 3.5 mm and a WOMAC Pain score of 40-90 points): As is apparent in FIG. 4B, and as expected, the subjects at risks treated only with placebo experience an increased loss of cartilage (mean change in cartilage thickness compared to baseline for this group at week 156 is of 0.07 mm), compared to placebo in the overall population of the study, see FIG. 1A). In contrast, subjects at risk treated with sprifermin injections (sprifermin 100 μg×4) experienced a limited loss of cartilage thickness during the period of treatment (mean change in cartilage thickness compared to baseline for this group at week 156 is of 0.03 mm). Thus, despite the propensity of the subjects at risk for rapid progression of the disease, the benefits of sprifermin in term of limitation of cartilage thinning observed in the study for the overall population of OA subjects. In all the figures, the term mJSW refers to the minimal joint space width in the whole knee.

Placebo Effect and Sprifermin Effect on WOMAC Total Score and Pain Score on the Overall Population of Patients Included in the Study:

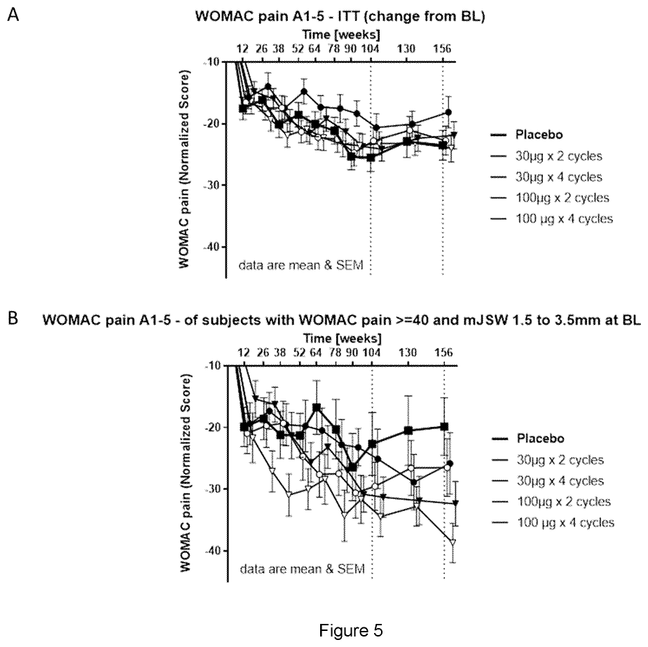

As is apparent in FIG. 5A, the change in WOMAC total scores is not statistically different in either placebo-treated subjects, and subjects treated with sprifermin, whether during the first 18 months when injections were made, or after the last injections. In all the figures, the term mJSW refers to the minimal joint space width in the whole knee.

Placebo Effect and Sprifermin Effect on WOMAC Total Score on Subjects at Risk (Minimal JSW of Between 1.5 and 3.5 mm and a WOMAC Pain Score of 40-90 Points)

As is apparent in FIG. 5B, the change in WOMAC total scores is statistically different in subjects treated with sprifermin (100 μg*4) compared to compared to placebo either placebo-treated subjects, by the end of the study (week 156), and thus despite the fact that the last injection is performed on week 78. The improvement in clinical symptoms as measured by the WOMAC score in the treated subject compared to placebo was unexpected, since these subjects are characterized by a non-acceptable pain at baseline and are expected to progress more rapidly towards more severe stages. Interestingly, in the treated subjects, the WOMAC total score continue to improve (negative change of the WOMAC total score) even after the last injection, in contrast with the placebo-treated subjects who experience a relative increase of their clinical symptoms in the same period (as shown by the change in WOMAC total score between week 78 and 156 for these subjects). This may reflect an indirect effect on sprifermin on the clinical symptoms of OA, at least on this specific subgroup. In all the figures, the term mJSW refers to the minimal joint space width in the whole knee.

Sprifermin Effect on WOMAC Total Score in Subjects at Risk

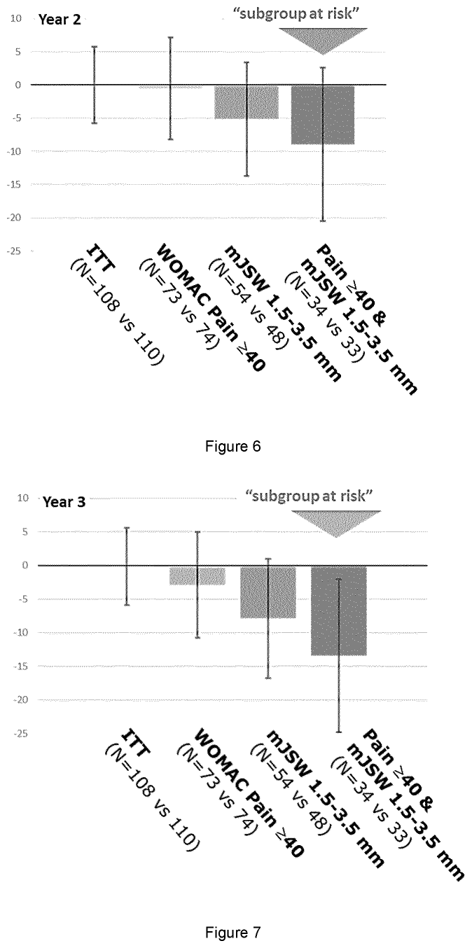

As is apparent in FIGS. 6 and 7, the extend of the effect of sprifermin on WOMAC Total score in the subjects at risk as defined herein is greater than in subjects presenting with only minimal JSW of between 1.5 and 3.5 mm or a WOMAC Pain score of 40 points or more at baseline, further suggesting that the effect on clinical symptoms observed is particularly improved specifically in subjects at risk of a rapid progression of the disease. In all the figures, the term mJSW refers to the minimal joint space width in the whole knee.

### REFERENCES

1) WO2008/023063
2) WO2004/032849
3) WO2014/023703
4) http://www.cartilage.org/_files/contentmanagement/ICRS_evaluation.pdf
5) Lotz, 2010, Arthritis research therapy, 12:211
6) Guermazi et al., 2015, Osteoarthritis Cartilage; 23(12): 2191-2198.
7) Pelletier et al., 2007, Arthritis Res Ther. 9(4):R74.
8) Wang et al., 2018, Arthritis Res Ther. 20: 250.
9) Ellsworth et al., 2002, Osteoarthritis and Cartilage, 10: 308-320
10) Shimoaka et al., 2002, J. Bio. Chem. 277(9):7493-7500
11) Gigout et al., 2017, Osteoarthritis and Cartilage, Osteoarthritis and Cartilage, 25(11):1858-1867.
12) The Merck Manual, 17^{th} edition, page 449
13) Bellamy et al., 1988, J. Rheumatology, 15:1833-1840
14) Wolfe, 1999, Rheumatology, 38:355-361
15) Hunter et al., 2009, Curr Opin Rheumatol.; 21(2):110-7.
16) Hawker et al., 2011, Arthritis Care & Research, 63(S11):S240-S252.
17) Williamson et al., 2005, J Clin Nurs.; 14(7):798-804.
18) Roos et al., 2003, Health Qual Life Outcomes; 1:17.
19) Collins et al., 2016, Osteoarthritis Cartilage. 24(8):1317-29.
20) Roos et al., 1998, Scand J Med Sci Sports.; 8(6):439-48.

1-11. (canceled)

12. A method of treating a subject having a cartilage disorder, wherein the subject presents with a risk of rapid progression of said cartilage disorder, comprising administering a FGF-18 compound comprising: a) amino acid residues 28-207 of SEQ ID NO:1, or b) SEQ ID NO: 2 to the subject, the FGF-18 compound limiting clinical symptoms associated with said cartilage disorder.

13. The method according to claim 12, wherein the clinical symptoms are selected from the group consisting of pain associated with said cartilage disorder, disability associated with said cartilage disorder and joint stiffness associated with said cartilage disorder.

14. The method according to claim 12, wherein the subject is considered as presenting with a risk of rapid progression of said cartilage disorder when said subject presents with:
(a) significant structural defects of the joint, said significant structural defects of the joint being selected from the group consisting of a minimal joint space width (miniJSW) of less than 3.5 mm and a KL grade of between 2 to 4;
(b) non-acceptable joint pain, said non-acceptable joint pain being selected from the group consisting of a joint pain corresponding to a WOMAC pain score of at least 35 points, a joint pain corresponding to a VAS pain score of 4 and higher (on a numeric scale) or 40 and higher (on a 100 mm scale), a joint pain corresponding to a NRS score of 4 and higher (on a 0-11 scale) and a joint pain corresponding to a KOOS score of 40 and above (on a 0-100 scale).

15. The method according to claim 12, wherein the cartilage disorder is selected from the group consisting of osteoarthritis, cartilage injury, fractures affecting joint cartilage or surgical procedures with impact on joint cartilage.

16. The method according to claim 12, wherein the FGF-18 compound is administered intraarticularly.

17. The method according to claim 12, wherein the FGF-18 compound is administered according to a dosing regimen comprising at least a treatment cycle of at least 2 administrations, said 2 administrations being separated by about 4, 5, 6, 7, 8, 9 or 10 days.

18. The method according to claim 12, wherein the FGF-18 compound is administered intraarticularly, at a dose of 100 μg per injection, once weekly for 3 weeks per treatment cycle, in a dosing regimen comprising at least four treatment cycles, said treatment cycles being separated by about 4 to 8 months.

19. A method for the treatment of clinical symptoms associated with a cartilage disorder in a subject having said cartilage disorder, wherein the subject presents with a risk of rapid progression of said cartilage disorder and a FGF-18 compound comprising: a) amino acid residues 28-207 of SEQ ID NO:1, orb) SEQ ID NO: 2 to the subject.

20. A method for selecting a subject having a cartilage disorder for inclusion in treatment, or clinical trial, with an active compound, based on the likelihood of their sensitivity to said treatment, comprising the steps of:
a) determining whether said subject presents with at least a significant structural defect of at least one joint, wherein the significant structural defect is selected from the group consisting of a minimal joint space width (miniJSW) of less than 3.5 mm, and a KL grade of 2 to 4, and;
b) obtaining an assessment of the level of joint pain of the subject, wherein the level of joint pain is assessed based on the WOMAC pain score, the VAS pain score, the NRS score or the KOOS score;
c) selecting the sensitive subjects as being suitable for said treatment or clinical trial.

21. A method of determining placebo effect in a clinical trial, wherein said clinical trial is related to the treatment of a cartilage disorder in a subject with an active compound, or during a treatment of a cartilage disorder with an active compound, the method comprising the steps of:
a) determining whether said subject presents with at least a significant structural defect of at least one joint, wherein the significant structural defect is selected from the group consisting of a minimal joint space width (miniJSW) of less than 3.5 mm and a KL grade of 2 to 4, and;
b) obtaining an assessment of the level of joint pain of the subject, wherein the level of joint pain is assessed based on the WOMAC pain score, the VAS pain score, the NRS score or the KOOS score;
c) determining from the result of steps a) and b) the placebo effect.
